(19) United States
(12) Patent Application Publication
(10) Pub. No.: US 2019/0316092 A1
(43) Pub. Date: Oct. 17, 2019

(54) METHODS FOR ISOLATING AND CULTURING LIVING CELLS USING METHOD OF PERMEABILIZING CELL MEMBRANE

(71) Applicant: SEOUL NATIONAL UNIVERSITY HOSPITAL, Seoul (KR)

(72) Inventors: Hyo-Soo Kim, Seoul (KR); Han-Mo Yang, Seoul (KR); Joo-Eun Lee, Seoul (KR); Ju-Young Kim, Gyeonggi-do (KR)

(21) Appl. No.: 16/382,267

(22) Filed: Apr. 12, 2019

(30) Foreign Application Priority Data
Apr. 13, 2018 (KR) 10-2018-0043354

(51) Int. Cl. C12N 5/077; C12N 5/078; C12N 5/074

(52) U.S. Cl. CPC C12N 5/0658; C12N 5/0696; C12N 5/0644; C12N 5/0661

(57) ABSTRACT

Provided are methods of isolating and culturing various cells in a living state including peripheral blood mononuclear cells (PBMCs) isolated from peripheral blood, which use a cell membrane permeabilization method. The methods use a streptococcal hemolytic exotoxin, which binds to cholesterol present in a cell membrane so as to make a pore therein, thereby allowing an exogenous protein to be permeated into the cell, and is a technique of isolating and culturing desired cells in a living state by probing a specific intracellular protein and performing flow cytometry (FACS). According to the methods, various intracellular proteins that could not previously be isolated and cultured from a patient's blood as well as various tissues may be targeted, and homogeneous cells may be obtained with high purity and high efficiency. Therefore, it is expected that the methods will highly contribute to many applications targeting a specific intracellular protein and research on mechanisms of various diseases and treatment thereof.

### CROSS-REFERENCE TO RELATED APPLICATION

This application claims priority to and the benefit of Korean Patent Application No. 2018-0043354, filed on Apr. 13, 2018, the disclosure of which is incorporated herein by reference in its entirety.

### BACKGROUND

### 1. Field of the Invention

The present invention relates to a technique of isolating and culturing cells in a living state using a method of permeabilizing a cell membrane.

### 2. Discussion of Related Art

Permeabilization of the cell membrane of a living cell is a method of forming a pore by binding to cholesterol commonly present in the cell membrane, and there are two different methods. The first method is a method using cholesterol-dependent cytolysins (CDCs), which bind to cholesterols present in the cell membrane so as to form pores therein. The CDCs are a superfamily of MACPFs, which are β-barrel pore-forming exotoxins mostly secreted from gram-positive bacteria. The second method is a method using non-ionic detergents, for example, digitonin. When a very small amount of digitonin is treated, it is bound with cholesterol of the cell membrane so as to selectively form a pore.

Among the CDCs, a streptococcal hemolytic exotoxin, i.e., Streptolysin O (SLO), is a single polypeptide chain with a size of 69 KD, which was found by E. W. Todd in 1939. The streptococcal hemolytic exotoxin is adsorbed onto a sterol (ergosterol, stigmasterol, cholestanol or the like) such as cholesterol, which is a main component of an animal cell membrane to melt a membrane with a bilayer structure, thereby forming a pore with a diameter of about 35 nm, and induces hemolysis in erythrocytes of various types of animals at different concentrations.

Meanwhile, since cell isolation technology has developed, various methods for transporting an exogenous protein into a specific cell have been studied. Representatively, the methods can be classified into 1) a protein transport method using exocytosis, 2) electroporation and 3) permeabilization. Exocytosis is the process of expelling substances from the cell membrane to an extracellular space without a damage through the fusion of an intracellular structure enveloped by a membrane with the cell membrane. While a protein may be transported into a cell by electroporation, to transport the target protein, the protein is pre-treated with, for example, a polymer matrix or fine particles, which gives rise to disadvantages of the change in a three-dimensional structure, a decrease in activity, and high costs. Conventionally, for isolation and culture of specific cells using fluorescence-activated cell sorting (FACS), only specific proteins present in the extracellular membrane were able to be used. In addition, to detect a specific protein in cells, the analysis and isolation of cells were made possible only when the cells were perforated to the extent of death with methanol or saponin and then fixed, but the isolated cells could not be cultured. In addition, to isolate cells in a living state, a probe such as a molecular beacon has been used, but such a conventional method has disadvantages of a high unit cost and amplification of a non-specific signal depending on a preparation method and a length. However, despite these problems, until now, there is no technique for effectively isolating and culturing cells in a living state, and a specific protein expressed in cells is abandoned or a circumventing experiment is being conducted with an indirect protein.

### PRIOR ART DOCUMENT

### Non-Specific Document

(Non-specific document 1) Journal of Pharmaceutical and Biomedical Analysis, Volume 128, 5 Sep. 2016, Pages 455-461. Atsushi Shoji et al., “Monitoring of cholesterol oxidation in a lipid bilayer membrane using Streptolysin O as a sensing and signal transduction element”

### SUMMARY OF THE INVENTION

Therefore, the inventors have focused on the following requirements for isolating and culturing cells in a living state using a specific intracellular protein.

First, cells should be alive when a pore is made in the cell membrane. In other words, a method of perforating a cell membrane as a method of conserving most of cell membrane should be used, and for this, the fastest and most gentle method should be used, thereby obtaining a large number of living cells.

Second, an exogenous protein introduced into the cell should accurately recognize and detect a target protein with high sensitivity and high efficiency.

Third, to more accurately isolate desired cells, proteins in the cell membrane as well as the intracellular proteins should be detected, and thus a protein and an introduction method that can simultaneously use various types of fluorescence should be used.

Next, the inventors developed a method of detecting a specific intracellular protein in living cells, and conducted analyses repeatedly to verify its accuracy. As a result, they have been found that target cells can be isolated and cultured in a living state by forming a pore in various cells using a streptococcal hemolytic exotoxin and introducing antibodies capable of detecting a specific intracellular protein, and therefore, the present invention was completed.

More specifically, the present invention is directed to providing a method of isolating cells in a living state using a specific intracellular protein as a marker by forming a pore in the cell membrane using a streptococcal hemolytic exotoxin.

However, technical problems to be resolved in the present invention are not limited to the above-described problems, and other problems which are not described herein will be fully understood by those of ordinary skill in the art from the following descriptions.

To attain the above-described object, the present invention provides a method of isolating target cells in a living state, which includes:

(s1) treating a test material including target cells with a streptococcal hemolytic exotoxin, i.e., SLO;

(s2) treating the test material with an antibody against a target intracellular protein of the target cells; and

(s3) isolating the target cells in a living state, which have a positive response to the antibody.

Preferably, the target cells include peripheral blood mononuclear cells (PBMCs), skeletal myoblasts (SMBs), smooth muscle cells (SMCs) and human induced pluripotent stem cells.

Preferably, in the step (s1), a treatment concentration of the streptococcal hemolytic exotoxin, i.e., SLO, is 5 U/ml or less.

Preferably, in the step (s2), the target intracellular protein is a marker protein in the target cells.

Preferably, the marker protein of the target cells includes calponin, smooth muscle actin (SMA), a nuclear factor of activated T-cells, cytoplasmic 1 (NFATc1) and dextran.

Preferably, the marker protein of the target cells is NANOG of human induced pluripotent stem cells or Tom20 of cardiomyocytes.

The present invention provides a method of isolating target cells in a living state, which includes:

(s1) treating a test material including target cells with a streptococcal hemolytic exotoxin, i.e., SLO;

(s2) treating the test material with an antibody against a target intracellular protein of the target cells; and

(s3) isolating the target cells in a living state, which have a positive response to the antibody through fluorescence-activated cell sorting (FACS) analysis.

Preferably, in the step (s2), a fluorescent dye-conjugated antibody is used.

Preferably, in the step (s2), an antibody against a target intracellular protein of the target cells is used as a primary antibody, and a fluorescent dye-conjugated secondary antibody is used.

Preferably, the fluorescent dye-conjugated secondary antibody has a size of 150 kDa or less.

Preferably, the fluorescent dye is one or more selected from the group consisting of FITC, PE, Alexa Fluor 488, DyLight 488, PerCP, PerCP-Cy5.5, Alexa Fluor 555, Alexa Fluor 633, Alexa Fluor 700, Alexa Fluor 405, Cy3, and Cy5.

Preferably, the method further includes regenerating a cell membrane by suspending the target cells in a calcium chloride (CaCl₂))-added cell culture.

Hereinafter, the present invention will be described.

In one aspect, the present invention provides a method of isolating cells in a living state with high purity and high efficiency.

More specifically, in one aspect, the present invention provides a method of isolating target cells in a living state, which includes the following steps:

(s1) treating a target material including target cells with a streptococcal hemolytic exotoxin, i.e., SLO;

(s2) treating the test material with an antibody against a target intracellular protein of the target cells; and (s3) isolating the target cells in a living state, which have a positive response to the antibody.

Each step will be described in detail below.

The present invention includes treating a test material including target cells with a streptococcal hemolytic exotoxin, i.e., SLO (Step s1).

In the present invention, the “test material” used herein may be any material that is expected to include target cells to be isolated without limitation, and may be a mixture of cells and a cell culture, other than the target cells. The test material may be isolated from animals including a human, but the present invention is not limited thereto, and the test material may include blood and tissue. The blood may include peripheral blood, which refers to blood circulating in the body of an animal, for example, a human, and is able to be variously extracted from blood vessels such as an artery, a vein, a peripheral blood vessel, etc.

In the present invention, the “target cells” used herein refer to cells to be isolated, and various types of cells known in the art may be target cells. According to an exemplary embodiment of the present invention, the target cells may include PBMCs, skeletal myoblasts (SMBs), and human induced pluripotent stem cells, but the present invention is not limited thereto. The “peripheral blood mononuclear cells (PBMCs)” used herein refer to mononuclear cells present in the peripheral blood, and include immune cells such as B cells, T cells, macrophages, dendritic cells, natural killer cells (NK cells), etc., and granulocytes such as basophils, eosinophils, neutrophils, etc.

In the step (s1) of the present invention, the treatment with the streptococcal hemolytic exotoxin, i.e., SLO may vary according to various factors including the type and content of a test material, the type and size of target cells, and the content of a sterol in the cell membrane, and a concentration of the treated streptococcal hemolytic exotoxin is preferably 5 U/ml or less. When the concentration is 5 U/ml or more, the necrosis or apoptosis of target cells may increase.

In the step (s1) of the present invention, by treating the streptococcal hemolytic exotoxin, a pore is made in the cell membrane of the target cells included in the test material, and thus the target cells have permeability.

In the present invention, the term “permeabilized” used herein means “having permeability” or “forming(making) a pore in a(the) cell membrane”, which may be interchangeably used unless particularly defined otherwise.

The present invention includes treating the test material with an antibody against a target intracellular protein of the target cells (step (s2)).

In the step (s1) of the present invention, the target cells achieve permeability by the treatment with the streptococcal hemolytic exotoxin. In addition, the target cells are alive.

In the present invention, in the step (s2), the target intracellular protein may be any protein present in the target cells without limitation, and various proteins may be used as a target protein depending on the isolation purpose or type of target cells. For example, if the purpose of isolating the target cells is to isolate only the target cells from a mixture including the target cells (e.g., a primary culture containing the target cells), a protein becoming a marker for the target cells may be used as a target protein. The target intracellular protein includes, for example, calponin, SMA, NFATc1 and dextran, but the present invention is not limited thereto. The NFATc1 is the nuclear factor of activated T cells, which is a representative transcription factor, and a marker for circulating multipotent stem cells (CiMSs). In addition, calponin and SMA are markers of skeletal myoblasts (SMBs) and smooth muscle cells (SMCs). In addition, a marker protein of the target cells, as a target intracellular protein, may be NANOG when the target cells are human induced pluripotent stem cells, or a target intracellular protein may be Tom20 when the target cells are cardiomyocytes. These are merely examples, and the present invention is not limited thereto.

In the present invention, in the step (s2), the antibody against the target intracellular protein is not limited as long as it can have an antigen-antibody reaction with the target intracellular protein, and various types of antibodies known in the art may be used. The antibodies can be modified in various ways according to purpose. For example, for effective isolation of the target cells, a marker may be bound to the antibody, and may include a fluorescent dye. Alternatively, for example, the antibody may be used as a carrier, and in this case, since a material to be transported to the target cells may be loaded in the antibody, the antibody may include a material to be transported to the target cells.

In the present invention, in the step (s2), in addition to the treatment with the antibody against the target intracellular protein, an antibody against an extracellular membrane protein may be further treated. Various methods known in the art, which can be used to isolate target cells to be isolated with high purity may be further included, but a method which cannot isolate target cells in a living state by inducing the death of the target cells is excluded.

The present invention includes isolating the target cells in a living state, which have a positive response to the antibody (Step (s3)).

In the present invention, the “positive response to an antibody” means that the antigen-antibody reaction between the target intracellular protein of target cells and an antibody, which indicates the presence of a target intracellular protein of the target cells. To verify the reaction, various methods known in the art may be used, and preferably, FACS analysis is used.

In the present invention, the “isolation of target cells” may be performed by various methods known in the art, and a suitable method may be selected in consideration of the type, size, and origin of the target cells.

The present invention provides a method of isolating target cells in a living state, which includes the following steps:

(s1) treating a test material including target cells with a streptococcal hemolytic exotoxin, i.e., SLO;

(s2) treating the test material with an antibody against a target intracellular protein of the target cells; and

(s3) isolating the target cells in a living state, which have a positive response to the antibody through fluorescence-activated cell sorting (FACS) analysis.

The steps (s1) and (s2) have been described above, and thus the descriptions will be omitted to avoid duplication. Some overlapping parts in the description of the step (s3) will be omitted by citing the previous description.

The present invention includes isolating the target cells in a living state, which have a positive response to the antibody through fluorescence-activated cell sorting (FACS) analysis (s3).

In the present invention, the FACS analysis is a type derived from flow cytometry. It provides a way to sort heterogeneously mixed cells based on specific light scattering and fluorescent characteristics of each type of cells. This is a useful scientific tool, and provides a fast, objective and quantitative record for the fluorescent signal of each type of cells.

To utilize the FACS analysis, in the step (s2), a fluorescent dye-conjugated antibody is preferably used, or in the step (s2), an antibody against a target intracellular protein of the target cells is used as a primary antibody, and a fluorescent dye-conjugated secondary antibody may also be used. More preferably, the fluorescent dye-conjugated secondary antibody has a size of 150 kDa or less.

The fluorescent dye may use one or more selected from the group consisting of FITC, PE, Alexa Fluor 488, DyLight 488, PerCP, PerCP-Cy5.5, Alexa Fluor 555, Alexa Fluor 633, Alexa Fluor 700, Alexa Fluor 405, Cy3 and Cy5, and other than these, various types of fluorescent dyes known in the art may be used. Preferably, when two or more proteins are used as target proteins in order to isolate the target cells, two or more types of antibodies may be used. Here, the fluorescent dyes conjugated to the two or more types of antibodies may show different colors. For example, FITC and Alexa Fluor 488 are green fluorescent dyes, DsRed and Cy3 are yellow fluorescent dyes, Alexa Fluor 635 and Cy5 are orange fluorescent dyes, and PerCP and Alexa Fluor 555 are red fluorescent dyes. A combination of four of these dyes may be used.

In addition to the step s3, the present invention may further include regenerating the cell membrane of the target cells. Since there is a pore in the cell membrane of the target cells by the treatment with the streptococcal hemolytic exotoxin, the present invention may further include a step of regenerating the cell membrane of the target cells to inhibit the death of the target cells and restore them to an original state. To regenerate the cell membrane of the target cells, a method of suspending the target cells in a calcium chloride (CaCl₂))-added cell culture is preferably used, but the present invention is not limited thereto. More preferably, an additional material necessary for the viability or culture of the target cells, for example, serum is suitably added to the cell culture.

The step of regenerating the cell membrane of the target cells may be included between the step (s2) and the step (s3), or after the step (s3). More specifically, before confirming the positive response to an antibody following the treatment of the target cells with an antibody in the step (s2), a method for regenerating the cell membrane may be performed, between the confirmation of a positive response to an antibody and the isolation of target cells, a method of regenerating the cell membrane may be performed, or after the corresponding target cells having a positive response to an antibody are isolated, a method of regenerating the cell membrane of target cells may be performed, but the present invention is not limited thereto. If needed, the method of regenerating the cell membrane of target cells may be performed in various steps.

In the present invention, in addition to the above-described steps, a step of culturing, subculturing or cryopreserving the isolated target cells may be further included, but the present invention is not limited thereto. Various processes known in the art additionally using the isolated target cells may be included.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1A shows the confocal imaging results and a graph for comparing FITC-dextran positive cells, after PBMCs are isolated from the peripheral blood of a donor and treated with 5 U of a streptococcal hemolytic exotoxin, and then permeabilized cells are treated with various sizes of FITC-dextran;

FIG. 1B shows the results of detecting the fluorescence of a variety of FITC-dextran, after PBMCs are isolated from the peripheral blood of a donor and treated with a streptococcal hemolytic exotoxin at various concentrations, using a flow cytometer;

FIG. 2A shows the apoptosis and necrosis analysis results (top), and graphs for comparing the death of cells in which the cell membrane is regenerated (bottom), after PBMCs are isolated from the peripheral blood of a donor and then the cell membranes of the cells permeabilized by the treatment with a streptococcal hemolytic exotoxin at various concentrations are regenerated, using a flow cytometer;

FIG. 2B shows cell death analysis results (top), and graphs for comparing the death of cells in which the cell membrane is regenerated (bottom), after PBMCs are isolated from the peripheral blood of a donor, and then the cell membranes of the cells permeabilized by the treatment with a streptococcal hemolytic exotoxin at various concentrations are regenerated and treated with PI, using a flow cytometer;

FIG. 3A shows graphs (top) of the comparative analysis of NFATc1 targeting an endomembrane between a group in which a target expressed in the endomembrane is treated with a flow cytometry fixation and permeabilization buffer kit (R&D, FC009, USA) and a BD fixation and permeabilization solution (554722, BD, USA) (saponin concentration: 0.02-0.1%) and a group treated with 5 U of a streptococcal hemolytic exotoxin, after PBMCs are isolated from the peripheral blood of a donor, and then the cells are killed, and a graph (down) showing that there is no difference in efficiency between the conventional method and a method targeting an endomembrane in a living state;

FIG. 3B shows graphs (top) of the comparative analysis of CiMS expression rates (top) between a group in which a target expressed in the endomembrane is treated with a flow cytometry fixation and permeabilization buffer kit (R&D, FC009, USA) and a BD fixation and permeabilization solution (554722, BD, USA) (saponin concentration: 0.02-0.1%) and a group treated with 5 U of a streptococcal hemolytic exotoxin, after PBMCs are isolated from the peripheral blood of a donor, and then the cells are killed, and a graph (down) showing that there is no difference in efficiency between the conventional method and the method of the present invention;

FIG. 4A shows graphs (left) for analyzing fluorescence expression rates and graphs (right) for comparatively analyzing the most suitable secondary antibodies, when skeletal myoblast cells are treated with a streptococcal hemolytic exotoxin, treated with representative markers, calponin and SMA, and then treated with various sizes of secondary antibodies;

FIG. 4B shows results obtained by isolating PBMCs from the peripheral blood of a donor, treating the cells with a streptococcal hemolytic exotoxin, treating the cells with an antibody targeting NFATc1 expressed in the cells, and then sorting NFATc1 positive and negative cells using a flow cytometer;

FIG. 4C shows that only calponin positive cells are isolated using a flow cytometer after culturing a rat artery using an enzyme reaction, treating the tissue with a streptococcal hemolytic exotoxin, and treating the tissue with calponin, which is a representative intracellular expression marker, to isolate vascular smooth muscle, and then cross-stained with smooth muscle actin to prove that the cells are vascular smooth muscle cells;

FIG. 4D shows that calponin positive cells are isolated and cultured using a flow cytometer, after skeletal myoblasts are treated with a streptococcal hemolytic exotoxin and treated with calponin, which is an intracellular expression marker;

FIG. 5 shows the results of confirming cell death when PBMCs are isolated from the peripheral blood of a donor, treated with a streptococcal hemolytic exotoxin, stained with dextran, subjected to flow cytometry to sort dextran positive and negative cells after isolating and culturing for 48 hours, and graphs for comparatively analyzing the cell death;

FIG. 6A is a set of live confocal images showing that human induced pluripotent stem cells are alive, after NANOG, which is a representative marker of a stem cell, is treated with a streptococcal hemolytic exotoxin;

FIG. 6B is a set of images proving that human induced pluripotent stem cells differentiate into human cardiomyocytes, are treated with a streptococcal hemolytic exotoxin, are stained with Tom20, which is a representative cytoplasmic marker of mitochondria, followed by beating; and

FIG. 7 schematically illustrates an overall method of the present invention.

### DETAILED DESCRIPTION OF EXEMPLARY EMBODIMENTS

Hereinafter, the present invention will be described in further detail with reference to examples. However, the following examples are merely exemplary, and thus the present invention is not limited to following examples.

### Example 1. Isolation of PBMCs from Peripheral Blood

10 cc of blood of a normal group was well mixed in 20 mL of phosphate-buffered saline (PBS, Invitrogen, NY, USA), and 12 mL of Ficoll-Paque (GE Health Care, Piscataway, N.J.) was slowly added thereto from the bottom so as to separate layers. Following centrifugation at 2,500 rpm for 30 minutes, the serum was removed from the uppermost layer of the four layers, and only PBMCs present in the middle of the layers were isolated using a pipette and mixed in PBS. Afterward, the PBMCs were washed twice by centrifugation at 1,800 rpm for 10 minutes, suspended in PBS at 1×10⁶/100 uL, and then dispensed into EP tubes.

### Example 2 . Permeability of Streptococcal Hemolytic Exotoxin (Streptolysin O; SLO)

An experiment for determining an exact concentration for making a pore in a cell membrane using a streptococcal hemolytic exotoxin (Streptolysin O; SLO; Sigma-Aldrich, MO, USA) was performed. In a permeabilization step, the isolated PBMCs or cell lines were centrifuged in cold PBS (Invitrogen, NY, USA) at 1,800 rpm for 5 minutes to collect the cells. 1×10⁶/100 μL of the cells were suspended in PBS, and dispensed into EP tubes. Each cell line was treated with a suitable concentration of a streptococcal hemolytic exotoxin (Streptolysin O; SLO) in a 37□ water bath for 50 minutes and then incubated on ice for 1 minute, 1 mL of PBS was added to the sample, and then the cells collected by centrifugation at 4□ for 3 minutes at 1,800 rpm were resuspended in an ATP regeneration solution. The ATP regeneration solution was prepared by adding 1 mM ATP, 10 mM creatine phosphate, 25 μg/mL creatine kinase, 100 μM GTP (Sigma-Aldrich, MO, USA) and 1 mM dNTP (Applied Biosystems, CA, USA) to PBS.

### Example 3 . Analysis of Permeabilization Efficiency Using Dextran-FITC

### Example 3-1. Analysis of Permeabilization Efficiency Using Confocal Imaging

To analyze permeabilization efficiency, uptake using various molecular sizes of fluorescein isothiocyanate (FITC)-dextran (Sigma-Aldrich, MO, USA) was confirmed by FACS and confocal imaging.

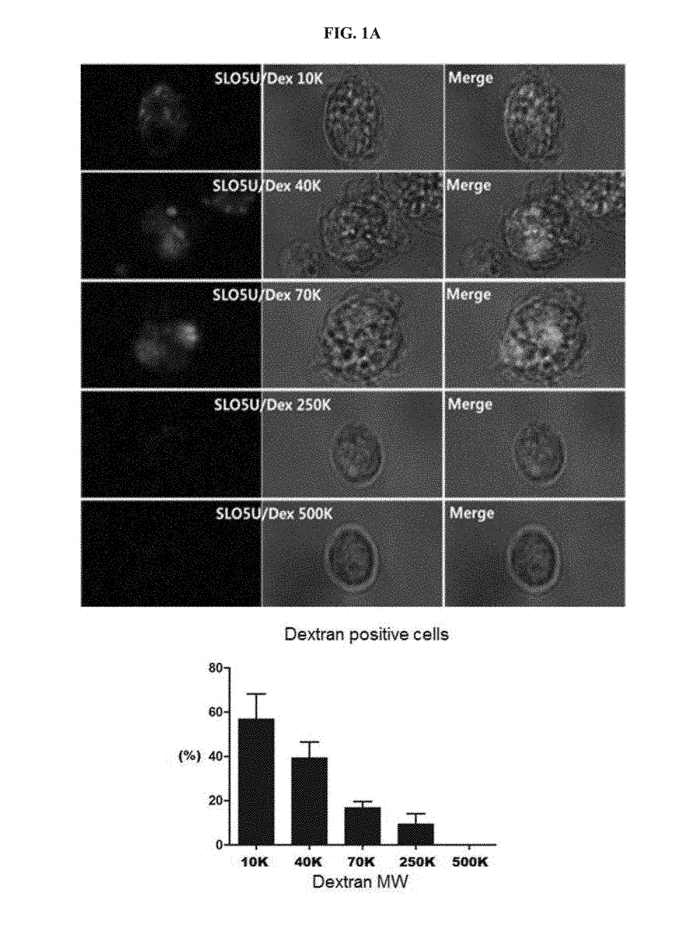

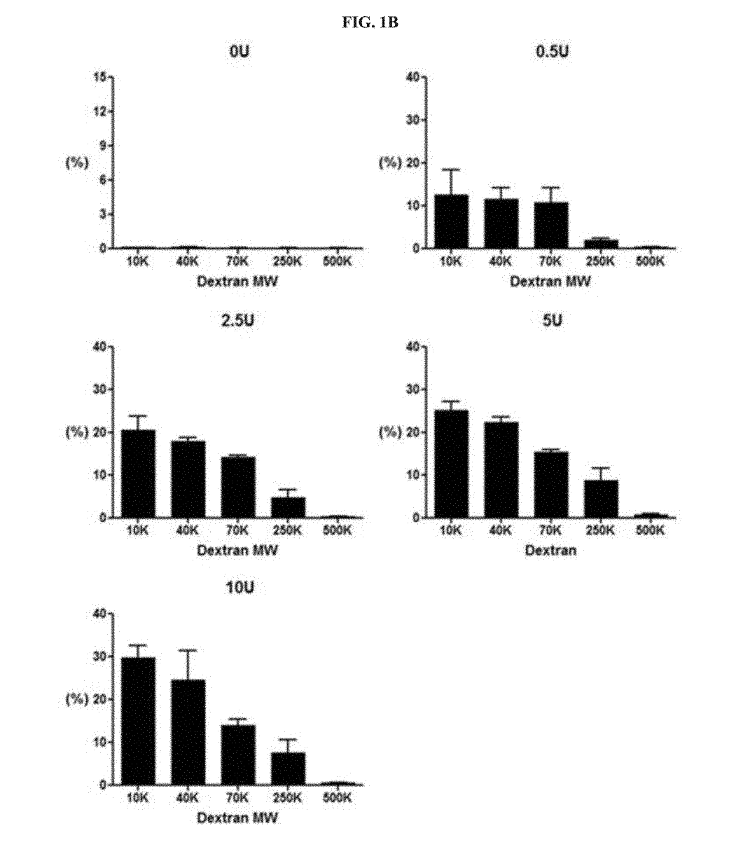

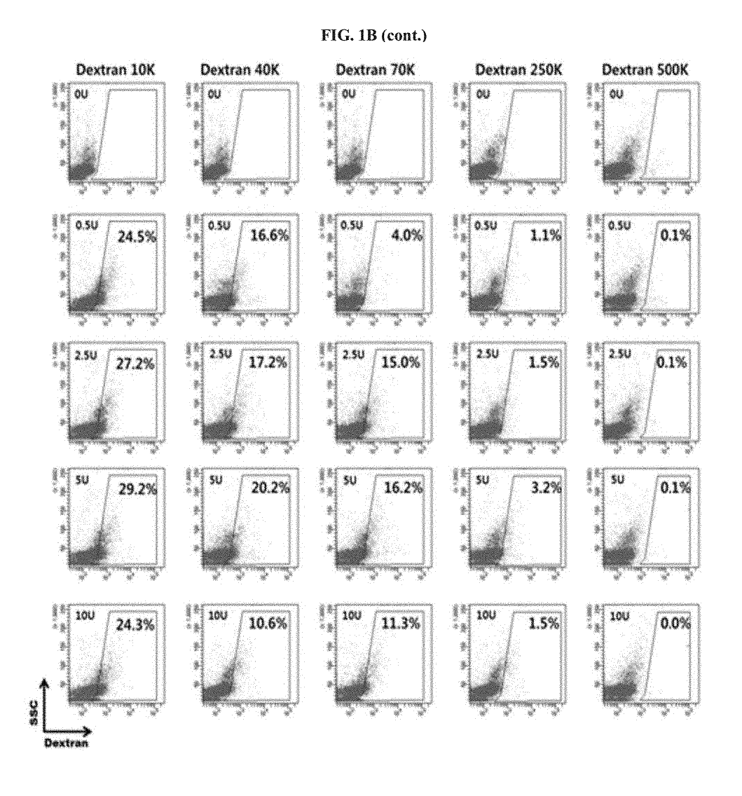

Specifically, the permeabilized cell line was dispensed into round-bottom tubes (BD Biosciences, CA, USA) at 5×10⁵/100 μL, and the cells were treated with FITC-dextran 10 KMW, 40 KMW, 70 KMW, 250 KMW, or 500 KMW (Sigma-Aldrich, MO, USA) by size, and incubated at 37□ for 1 hour. After staining, to regenerate the cell membrane of a cell, 2 mM CaCl₂) was added to the same volume of the cell culture solution, and the cells were incubated at 37□ for 2 hours and centrifuged at 4□ for 3 minutes at 1,800 rpm, followed by washing. The cells were resuspended in the cell culture solution to which 2 mM CaCl₂) was added and mounted on a confocal dish (Ibidi, Germany), fluorescence was visualized using a confocal laser scanning microscope 710 (Zeiss, Germany) and analyzed using Zeiss Zen software, and FITC positive cells were statistically analyzed using Image J. It was confirmed that, according to the above-described method, up to FITC-dextran 250K among those of various molecular sizes was permeated into the cells using SLO. FIG. 1A shows confocal images of FITC positive cells, and the result is shown by a graph.

### Example 3-2. Permeabilization Efficiency Assay Using FACS

To see permeabilization efficiency, using various molecular sizes of the FITC-dextran (Sigma-Aldrich, MO, USA), uptake was confirmed by FACS. Specifically, the permeabilized cell line was dispensed into round-bottom tubes (BD Biosciences, CA, USA) at 5×10⁵/100 FITC-dextran 10 KMW, 40 KMW, 70 KMW, 250 KMW or 500 KMW (Sigma-Aldrich, MO, USA) were added to the cells by size, and the cells were incubated at 37□ for 1 hour and stained. To regenerate the cell membrane of a cell, 2 mM CaCl₂) was added to the same volume of the cell culture solution, and the cells were incubated at 37□ for 2 hours, and centrifuged at 4□ for 3 minutes at 1,800 rpm, followed by washing. The cells were resuspended in a 2 mM CaCl₂)-added cell culture solution, and a fluorescent material was detected using FACS Canto II (BD Biosciences, CA, USA) (all data measured by FACS was analyzed using BD FACSDiva software). While adding various concentrations of SLO, various molecular sizes of FITC-dextran were uptaken, and the ratio of permeated positive cells is shown in FIG. 1B.

### Example 4 . Method of Confirming Cell Death of Cell Line Treated with Streptococcal Hemolytic Exotoxin (Streptolysin O; SLO)

### Example 4-1. Method of Confirming Necrosis and Apoptosis

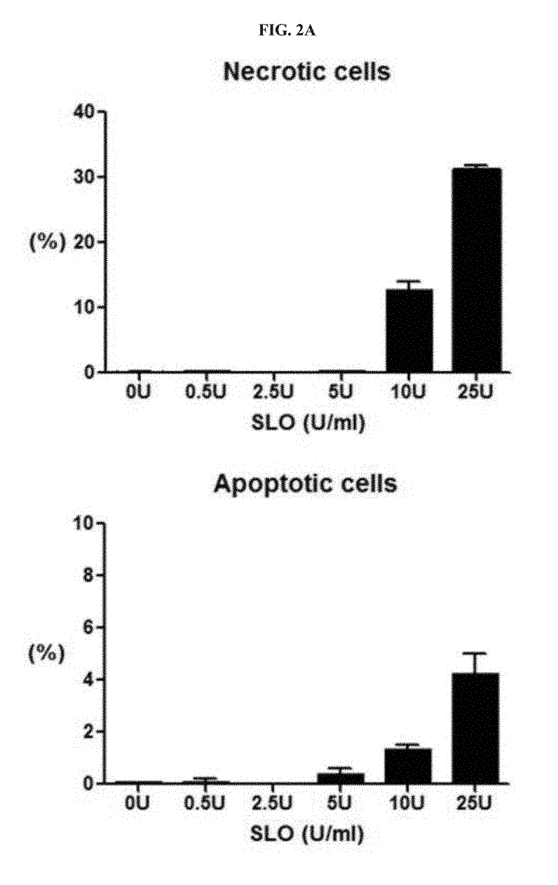

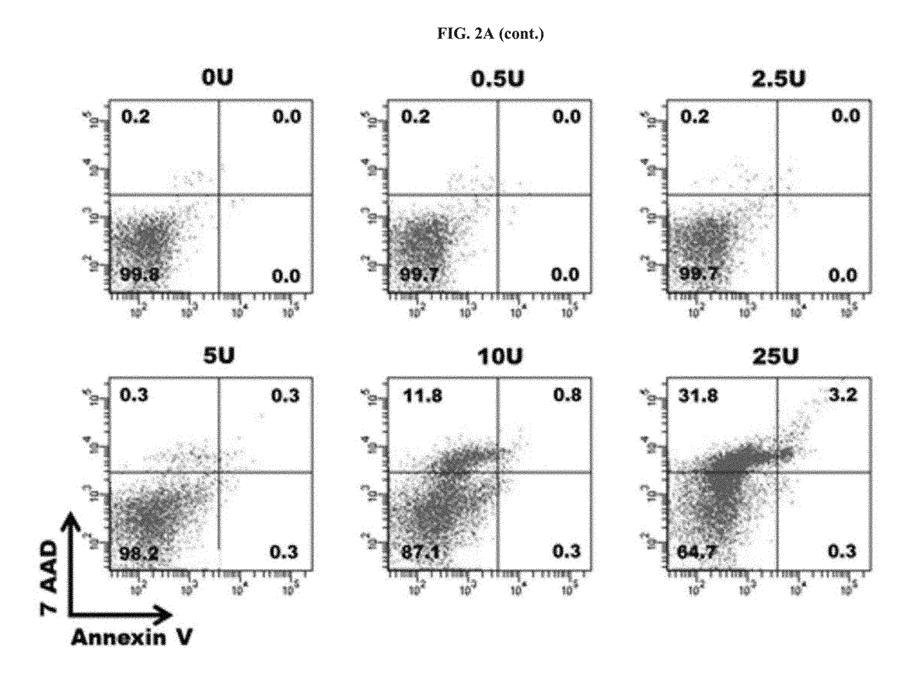

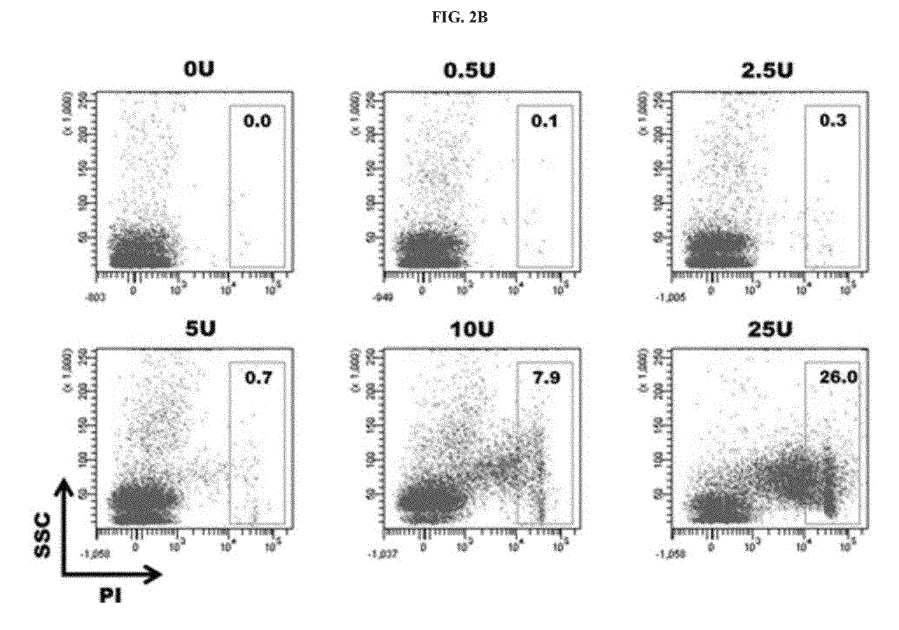

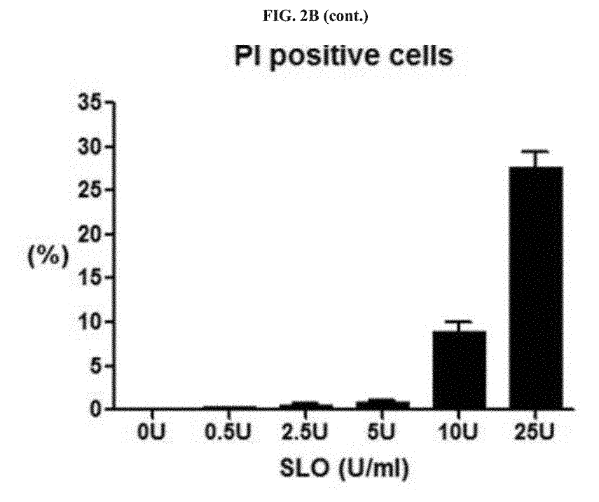

Permeabilized cells were resuspended in cold PBS (Invitrogen, NY, USA), and to regenerate the cell membrane of a cell, 2 mM CaCl₂) was added to the same volume of the cell culture solution, and then the cells were incubated at 37□ for 2 hours, and centrifuged at 4□ for 3 minutes at 1,800 rpm, followed by washing. The cells were resuspended in the 2 mM CaCl₂)-added cell culture solution, and dispensed into round-bottom tubes (BD Biosciences, CA, USA) at 5×10⁵/100 μL. The cells were mixed with 7AAD (BD Biosciences, CA. USA) and ANNEXIN V (BD Biosciences, CA. USA), cultured at room temperature for 15 minutes, and stained. Afterward, the cells were centrifuged in 2.5% fetal bovine serum, (FBS 16000, Gibco, NY, USA)-added PBS (Invitrogen, NY, USA) at 1,800 rpm for 5 minutes for washing, resuspended in 100 μL of washing buffer, and analyzed using FACS Canto II (BD Biosciences, CA, USA). All data measured by FACS was analyzed using BD FACSDiva software. As shown in FIG. 2A, it can be seen that necrotic and apoptotic cells increased according to an increase in the concentration of SLO.

### Example 4-2. PI Staining Method

Permeabilized cells were resuspended in cold PBS (Invitrogen, NY, USA), and to regenerate the cell membrane of a cell, 2 mM CaCl₂) was added to the same volume of the cell culture solution, and then the cells were incubated at 37□ for 2 hours, and centrifuged at 4□ for 3 minutes at 1,800 rpm, followed by washing. The cells were resuspended in the 2 mM CaCl₂)-added cell culture solution, and dispensed into round-bottom tubes (BD Biosciences, CA, USA) at 5×10⁵/100 μL. The cells were mixed with propidium iodine (PI, BD Biosciences, CA. USA), incubated at room temperature for 15 minutes and stained. Then, the cells were centrifuged in 2.5% fetal bovine serum (FBS 16000, Gibco, NY, USA)-added PBS (Invitrogen, NY, USA) at 1,800 rpm for 5 minutes for washing, resuspended in 100 μL of washing buffer, and analyzed using FACS Canto II (BD Biosciences, CA, USA). All data measured by FACS was analyzed using BD FACSDiva software. As shown in FIG. 2B, it can be confirmed that as the SLO concentration increased, PI positive cells increased, and it can be seen that the most suitable concentration was 5 U.

### Example 5. Methods of FACS and Sorting for SLO-Treated Cell Line

### Example 5-1. Comparative Analysis of Difference in Expression of NFATc1 and CiMS Cells in PBMCs According to Permeabilization Buffer

Using PBMCs permeabilized using SLO and commercially available R&D, a BD Permeabilization buffer, a flow cytometry fixation and permeabilization buffer kit (R&D, FC009, USA) (saponin concentration: 0.02-0.1%) and a BD fixation and permeabilization solution (554722, BD, USA), the permeabilized PBMCs were resuspended in cold PBS (Invitrogen, NY, USA), and the cells were dispensed into round-bottom tubes (BD Biosciences, CA, USA) at 5×10⁵/100 μL. 5 μL each of allophycocyanin (APC) conjugated-mouse anti-human CD45 (BD Biosciences, CA, USA), APC conjugated-mouse anti-human CD3 (BD Biosciences, CA, USA), Alexa 488 conjugated-anti-human NFATc1 (Santa Cruz Biotechnology, CA, USA) and phycoerythrin (PE) conjugated-mouse anti-human CD31 (BD Biosciences, CA, USA) was added to each cell-dispensed tube, and the cells were incubated at room temperature for 30 minutes and stained. Afterward, to regenerate the cell membrane of the cell, 2 mM CaCl₂) was added to the same volume of the cell culture solution, and the cells were incubated at 37□ for 2 hours, and centrifuged in 2.5% fetal bovine serum (FBS 16000, Gibco, NY, USA)-added PBS (Invitrogen, NY, USA) at 1,800 rpm for 5 minutes for washing. The collected cells were resuspended in the 2 mM CaCl₂)-added cell culture solution, and analyzed using FACS Canto II.

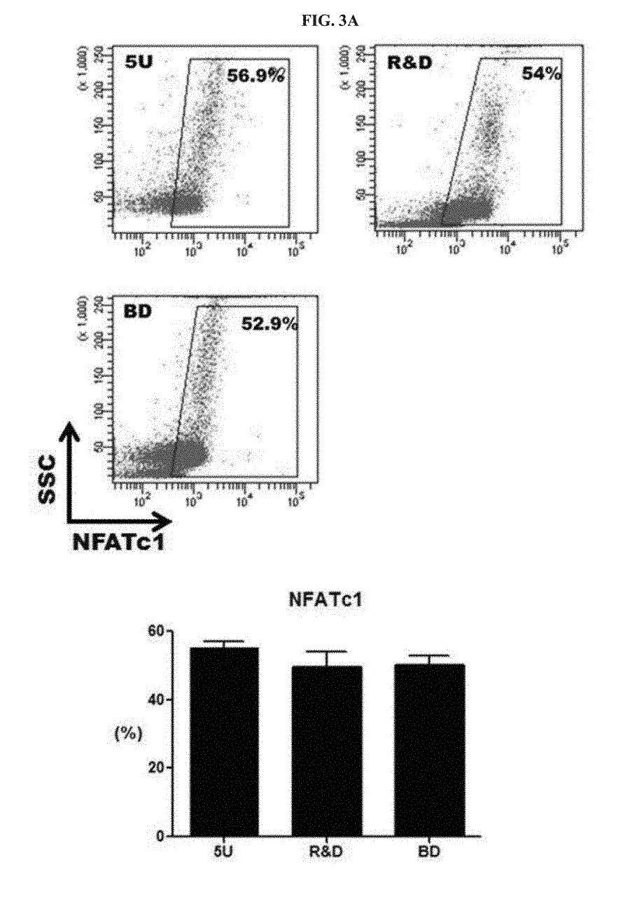

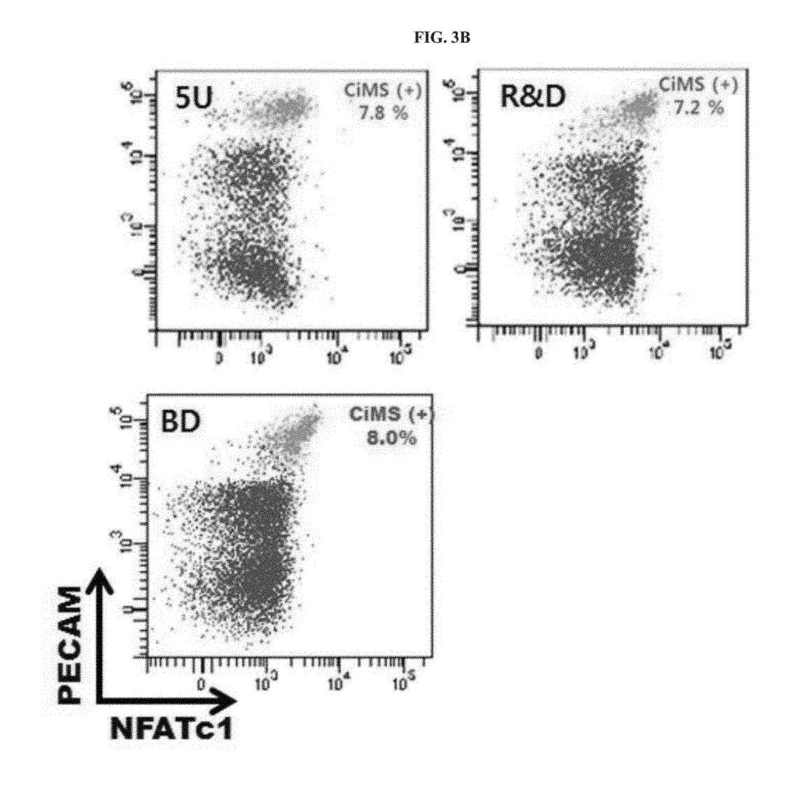

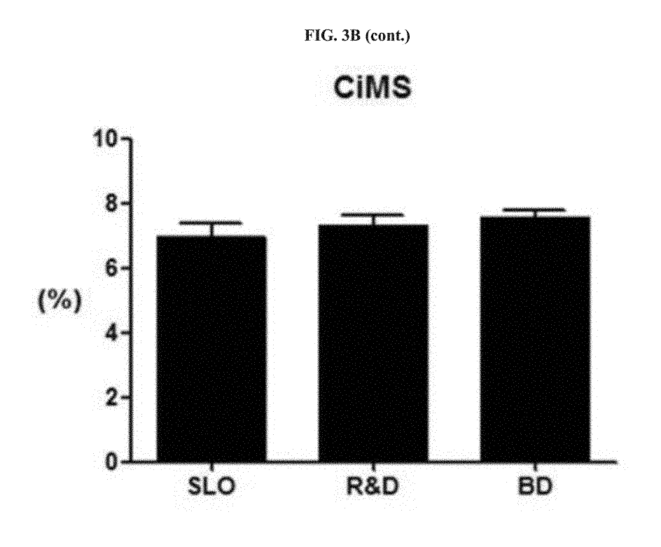

As a result, as shown in FIG. 3A, under conditions in which the commercially available permeabilization buffer and 5 U SLO are used, it was proven that there was no difference in expression of NFATc1 positive cells, and as shown in FIG. 3B, it was demonstrated that, under conditions in which the commercially available permeabilization buffer and 5 U SLO are used, there was no difference in expression level of PBMC-derived circulatory multipotent stem cells (CiMS cells) (NFATc1+/CD31+/CD3+/CD45+), either.

### Example 5-2. Calponin and SMAFACS for SMB Cell Line

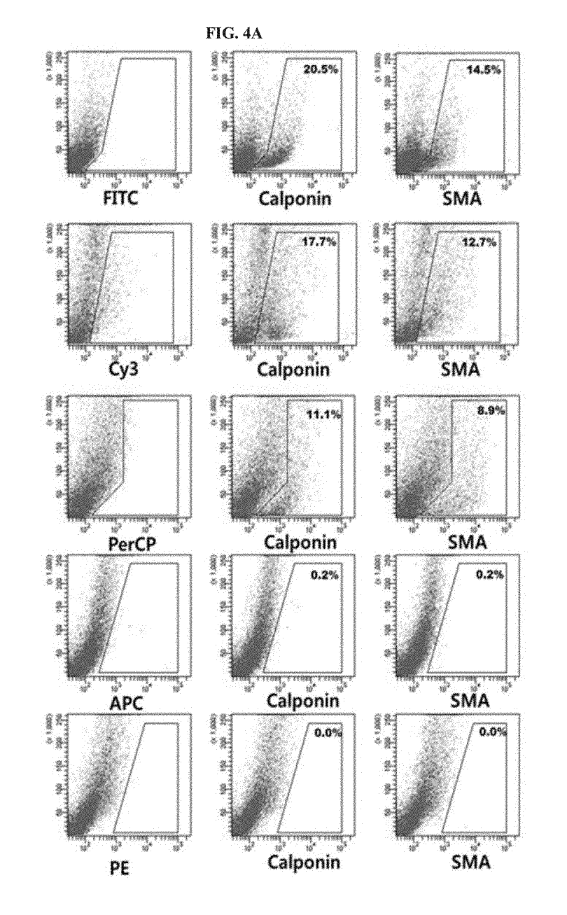

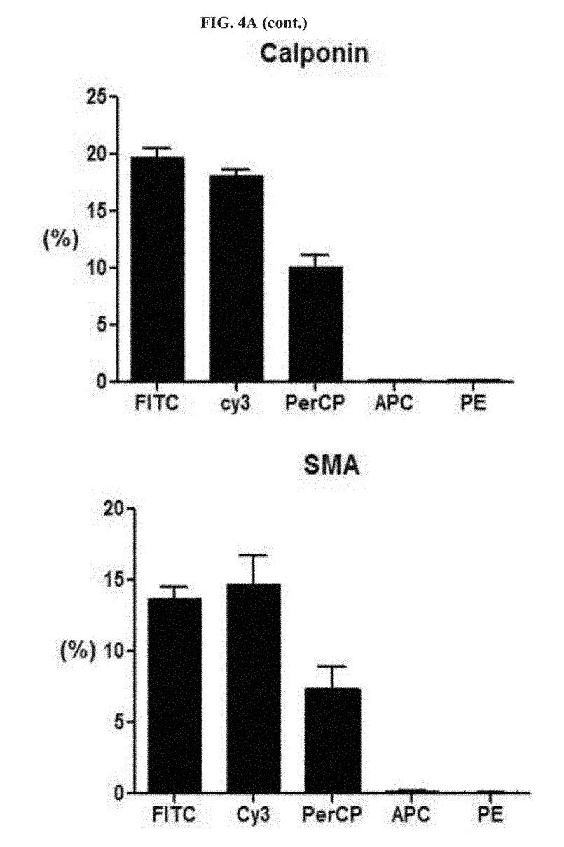

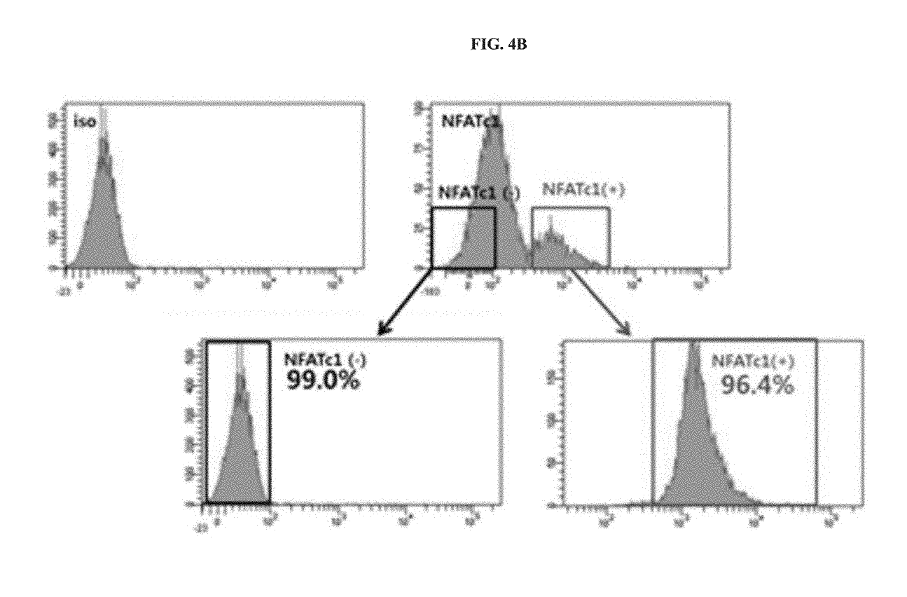

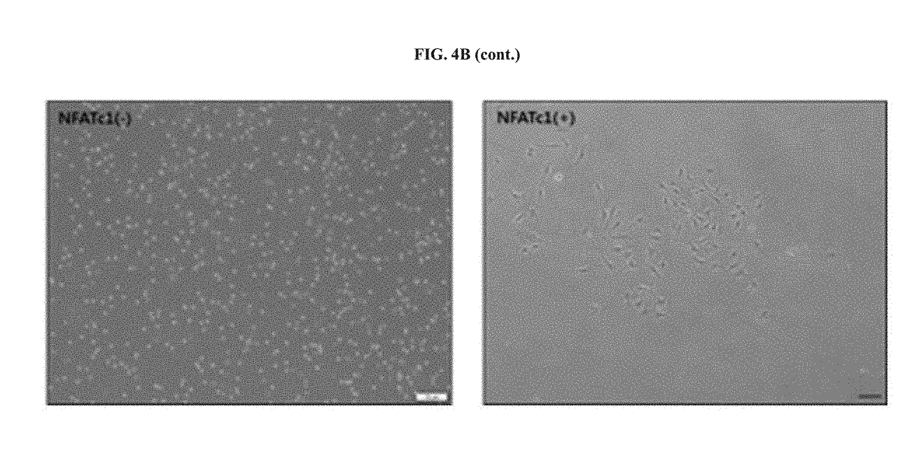

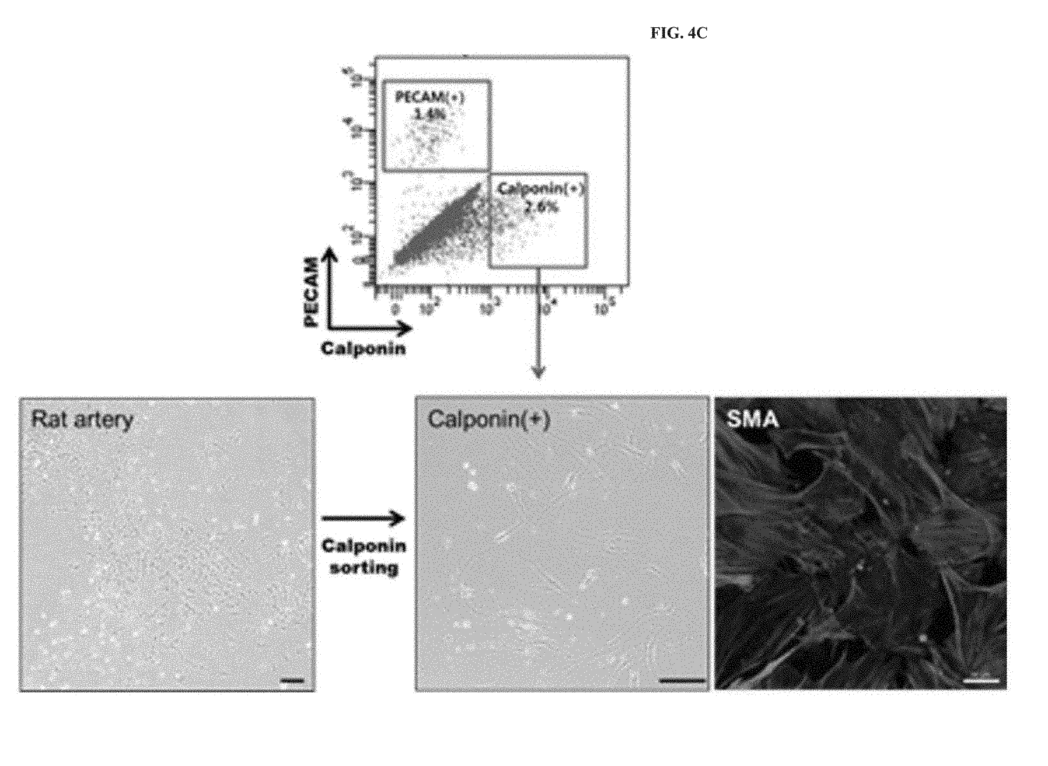

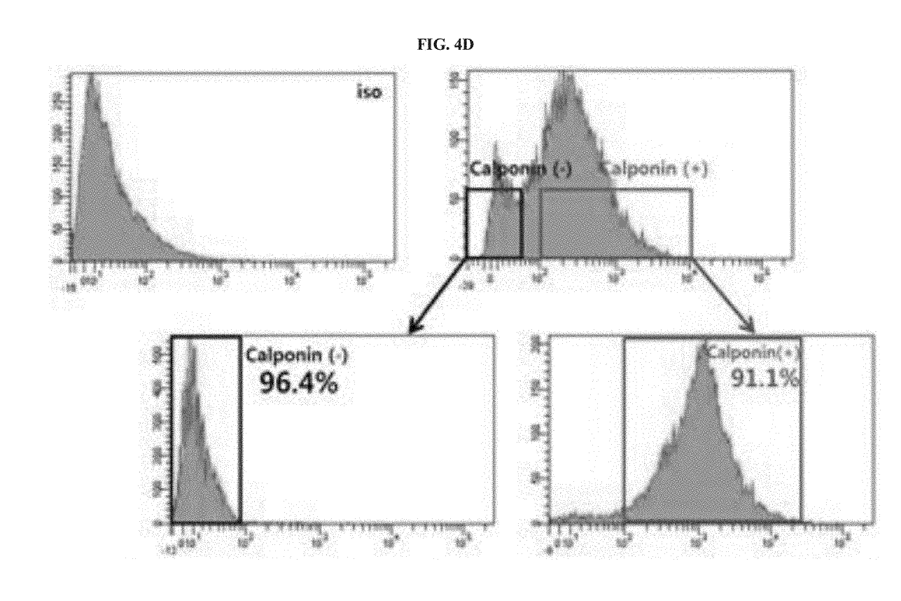

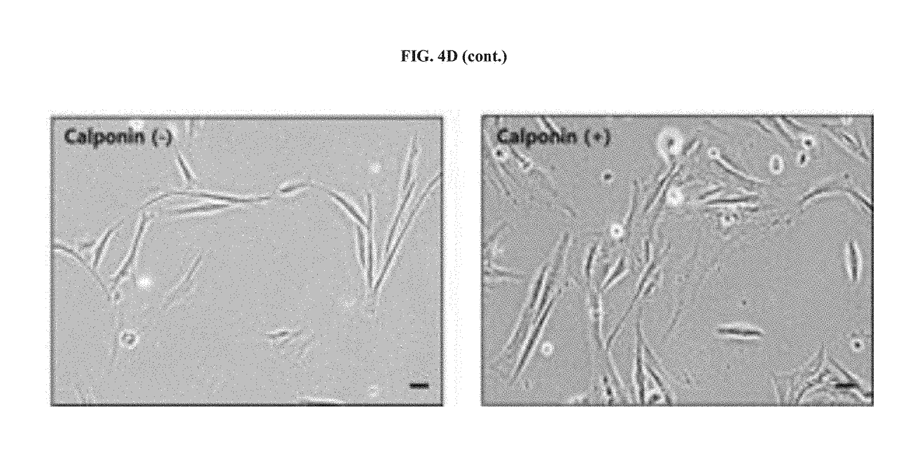

Permeabilized SMBs were resuspended in cold PBS (Invitrogen, NY, USA), and the cells were dispensed into round-bottom tubes (BD Biosciences, CA, USA) at 5×10⁵/100 μL. 5 μL each of mouse-anti-calponin (Sigma) and mouse anti-SMA (Abcam) was added to each tube, and incubated at room temperature for 30 minutes. After the first staining, as a secondary antibody, 2 μL each of a fluorescein isothiocyanate (FITC)-conjugated antibody (BD Biosciences, CA, USA), a PerCP-conjugated antibody (BD Biosciences, CA, USA), and a Cy3-conjugated antibody (BD Biosciences, CA, USA) were added to the resulting cells, and then incubated at room temperature for 30 minutes. After the second staining, to regenerate the cell membrane of each cell, 2 mM CaCl₂) was added to the same volume of SK-GMV (Lonza), the cells were incubated at 37□ for 2 hours, and centrifuged in 2.5% fetal bovine serum (FBS 16000, Gibco, NY, USA)-added PBS (Invitrogen, NY, USA) at 1,800 rpm for 5 minutes for washing. The collected cells were resuspended in 2 mM CaCl₂)-added SK-GMV (Lonza), analyzed using FACS Canto II and then represented by a graph shown in FIG. 4A. When the size of the secondary fluorescent antibody was 150 kDa or more (APC, PE), it did not permeate, showing that it is not suitable for the analysis method using SLO.

### Example 5-3. Detection of NFATc1 Positive Cells in PBMCs

Permeabilized PBMCs were resuspended in cold PBS (Invitrogen, NY, USA), and the cells were dispensed into round-bottom tubes (BD Biosciences, CA, USA) at 5×10⁵/100 μL. 5 μL of Alexa 488-conjugated anti-human-NFATc1 (Santa Cruz Biotechnology, CA, USA) was added to each tube, incubated at room temperature for 30 minutes, and stained. Afterward, to regenerate the cell membrane of the cell, 2 mM CaCl₂ was added to the same volume of cell culture solution, cells were incubated at 37□ for 2 hours, and centrifuged in 2.5% fetal bovine serum (FBS 16000, Gibco, NY, USA)-added PBS (Invitrogen, NY, USA) at 1,800 rpm for 5 minutes for washing. The collected cells were resuspended in 2 mM CaCl₂-added cell culture solution, NFATc1-stained cells were filtrated through a mesh, and NFATc1 positive and negative cells were concentrated in PBS using a FACSAria cell sorter (BD Biosciences, CA, USA) and then centrifuged at 1,800 rpm for 10 minutes. The collected cells were resuspended in 2 mM CaCl₂-added EGM2-MV (Lonza, USA), and seeded in a 10 μg/mL fibronectin (Sigma-Aldrich, MO, USA)-coated Corning 6-well plate. The medium was changed daily, and after 5 days, the cells were able to be identified as shown in FIG. 4B.

### Example 5-4. Method of Culturing Primary Rat Artery

After six-week-old rats were killed by inhalation anesthesia with CO₂, arteries of the rats were harvested and put into cold PBS, and then cells were obtained by a method using an enzyme reaction.

More specifically, after blood removal, the harvested rat arteries were cut into small pieces, added to a conical flask containing 5 mL of 0.05% collagenase type II (Invitrogen, NY, USA) in high glucose DMEM, and left at 37□ on a shaking incubator for 30 minutes. To terminate enzyme treatment, 10% FBS and the sample were suspended and centrifuged at 1000 rpm for 10 minutes. The collected cells were resuspended in DMEM, and centrifuged again for washing. The collected cells were seeded in a 1.5% gelatin (Sigma-Aldrich, MO, USA)-coated Corning® 60-mm dish containing high glucose DMEM which contains 10% FBS and antibiotics, and the medium was changed every other day. After seven days, when mixed cells were observed, they were subjected to cell permeabilization using SLO by the same method as described in Example 5-2, and as shown in FIG. 4C, only calponin positive cells were sorted and grown.

### Example 5-4-1. Immunocytochemistry

To confirm that the cells sorted in FIG. 4C were calponin positive cells, the cells were seeded in a 35-mm confocal dish (Ibidi, Germany), 1.5 mL of cold 100% methanol was added thereto, and the cells were maintained at −20□ for 10 minutes. The cells fixed with methanol were washed with a washing buffer containing 0.05% Tween 20 in PBS twice, and to reduce a non-specific reaction, 0.1% BSA was mixed in PBS, and a blocking buffer filtrated through a 0.22-mm mesh was added thereto, followed by maintaining at room temperature for 30 minutes. Mouse anti-SMA (rat smooth muscle actin; Abcam) antibodies were suspended in a 1:200 antibody diluent (Invitrogen, NY, USA) and then the resulting suspension was added to the blocked cells, followed by maintaining at 4□ overnight. After the first staining, the cells were washed with a washing buffer containing 0.05% Tween 20 in PBS three times, a secondary antibody, i.e., Alexa Fluor 488 anti-mouse IgG, was suspended in a 1:200 antibody diluent (Invitrogen, NY, USA), and then the resulting suspension was added to the cells, followed by maintaining at room temperature for 1 hour. After the second staining, the cells were washed with a washing buffer containing 0.05% Tween 20 in PBS three times, and to stain the nucleus, and DAPI (Sigma-Aldrich) was suspended in a 1:1000 antibody diluent (Invitrogen, NY, USA), and then the resulting suspension was added to the cells, followed by maintaining at room temperature for 10 minutes. The nucleus-stained sample was treated with mounting media, covered with a cover glass, observed using a confocal laser scanning microscope 710 (Zeiss, Germany) to visualize fluorescence, and then analyzed using Zeiss Zen software (SMA-stained image of FIG. 4C).

### Example 5-4-2. Detection of Calponin and CD31 in SMB Cell Line

SMB cells were resuspended in cold PBS (Invitrogen, NY, USA), and dispensed into round-bottom tubes (BD Biosciences, CA, USA) at 5×10⁵/100 μL. 5 μL each of mouse-anti-calponin (Sigma) and PE-conjugated rat CD31 (BD Biosciences, CA, USA) was added to each tube, and incubated at room temperature for 30 minutes. After the staining, to regenerate the cell membrane of the cell, 2 mM CaCl₂) was added to the same volume of high glucose DMEM (Invitrogen, NY, USA) containing 10% fetal bovine serum (FBS 16000, Gibco, NY, USA), the cells were incubated at 37□ for 2 hours, and centrifuged in 2.5% fetal bovine serum (FBS 16000, Gibco, NY, USA)-added PBS (Invitrogen, NY, USA) at 1,800 rpm for 5 minutes for washing. The collected cells were resuspended in 2 mM CaCl₂)-added high glucose DMEM (Invitrogen, NY, USA) containing 10% fetal bovine serum (FBS 16000, Gibco, NY, USA), the stained cells were filtrated through a mesh, and then calponin positive cells were concentrated in PBS using a FACSAria cell sorter (BD Biosciences, CA, USA) and then centrifuged at 1,800 rpm for 10 minutes. The collected cells were resuspended in 2 mM CaCl₂)-added high glucose DMEM (Invitrogen, NY, USA) containing 10% fetal bovine serum (FBS 16000, Gibco, NY, USA), and seeded in a 1.5% gelatin (Sigma-Aldrich, MO, USA)-coated Corning® 6-well plate. The medium was changed daily, and after 10 days, as shown in FIG. 4D, it was observed that, compared with calponin negative cells, in calponin positive cells, actin was more highly formed, showing that the calponin positive cells were successfully isolated and cultured.

### Example 5-3. Detection of Dextran and Confirmation of Cell Death in PBMCs

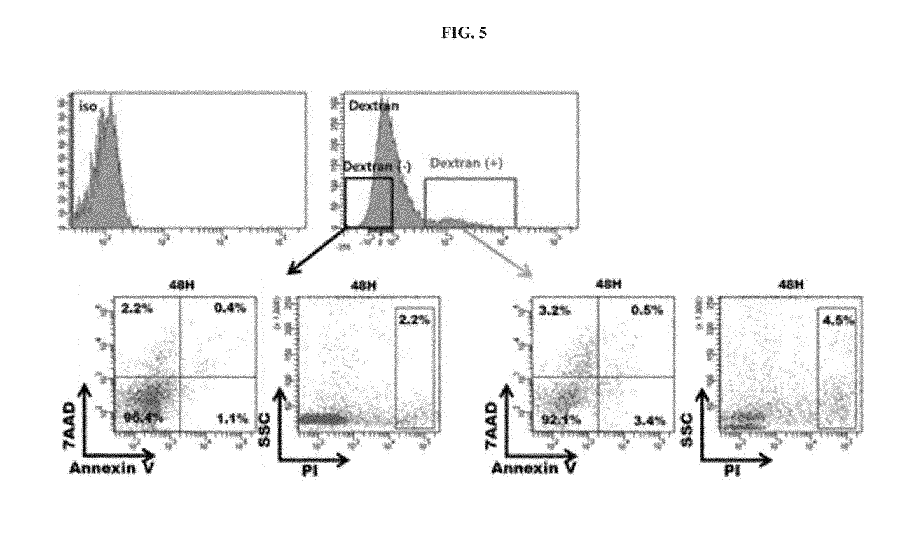

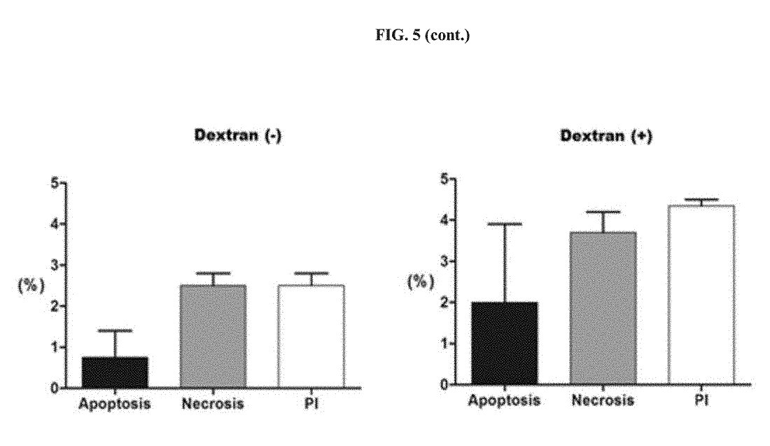

Permeabilized PBMCs were resuspended in cold PBS (Invitrogen, NY, USA), and the cells were dispensed into round-bottom tubes (BD Biosciences, CA, USA) at 5×10⁵/100 μL. 5 μL each of FITC conjugated-Dextran (Invitrogen, NY, USA) was added to each tube, and incubated at room temperature for 30 minutes. After the staining, to regenerate the cell membrane of the cell, 2 mM CaCl₂) was added to the same volume of a cell culture solution, the cells were incubated at 37□ for 2 hours, and centrifuged in 2.5% fetal bovine serum (FBS 16000, Gibco, NY, USA)-added PBS (Invitrogen, NY, USA) at 1,800 rpm for 5 minutes for washing. The collected cells were resuspended in 2 mM CaCl₂)-added EGM2-MV (Lonza, USA) and filtrated through a mesh, and dextran positive and negative cells were concentrated in PBS using a FACSAria cell sorter (BD Biosciences, CA, USA) and centrifuged at 1,800 rpm for 10 minutes. The collected cells were resuspended in 2 mM CaCl₂)-added EGM2-MV (Lonza, USA) and seeded in a 10 μg/mL fibronectin (Sigma-Aldrich, MO, USA)-coated Corning® 6-well plate, and after 48 hours, the cell line was analyzed by the method of confirming the cell death of a streptococcal hemolytic exotoxin (Streptolysin 0; SLO)-treated cell line, described in Example 4. As a result, as shown in FIG. 5, like dextran negative cells, in dextran positive cells, ratios of necrotic and apoptotic cells and PI positive cells were merely less than 5%, showing that dextran has substantially no effect on cell death.

### EXAMPLE 6

### Preparation of Induced Pluripotent Stem Cells Using Fibroblasts

### Example 6-1. Preparation of Retrovirus and Human Induced Pluripotent Stem Cells and NANOG Fluorescent Staining

Retroviruses expressing four genes, called Yamanaka factors, such as SOX2, c-MYC, OCT4 and KLF4, respectively, were prepared to be used for the preparation of induced pluripotent stem cells. To this end, 293T cells were cultured in high glucose DMEM containing 10% FBS and an antibiotic until reaching 90% of the area of a culture container. Meanwhile, 800 μL of basal DMEM was dispensed into each 1.5-mL Eppendorf tube, a plasmid in which one of the four genes prepared herein was cloned and packaging vectors, i.e., pVSV-G and pGag-Pol, were added at 10 μg/plasmid, 60 μL of 1 mg/mL polyethyleneimine (PEI) stock was added and well mixed, and the cell mixture was maintained at room temperature for 30 minutes. During this process, to increase transfection efficiency, the 293T cells were washed with 5 mL of basal DMEM twice, and then 10 mL of high glucose DMEM (containing 10% FBS) without an antibiotic was added thereto, and then the cells were stored in a 37□ incubator in a state in which an antibiotic was removed. Thirty minutes after the preparation of a plasmid DNA-PEI mixed solution, the antibiotic-removed 293T cells were extracted, and each plasmid DNA-PEI mixed solution per gene type was gently stirred once using a pipette and then dropped on the 293T cells for transfection. After 18 hours, the transfected 293T cells were taken out from the incubator, heated to 37□, and rinsed twice with 5 mL of basal DMEM to remove the excess plasmid DNA-PEI mixed solution. 10 mL of fresh high glucose DMEM containing 10% FBS and an antibiotic was added to the cells, and incubated in a 37□ incubator. Here, this process was very carefully performed so as not to detach the cells. After 48 hours, to recover the produced retroviruses, only the culture of 293T cells was collected in a 15 mL tube and centrifuged at 2500 rpm for 15 minutes, and then only the supernatant was harvested so that detached cells and debris, which were precipitated on the bottom, were not included, filtrated through a 0.22-μm filter, and subjected to ultracentrifugation at 4□ for 1.5 hours at 25,000 rpm to concentrate the retroviruses included in the supernatant. The retroviruses expressing four different genes, which were precipitated in pellets were resuspended in 100 μL of EBM-2, and stored at −70□ before use. The concentrated Yamanaka 4F were inoculated into fibroblasts, thereby preparing human induced pluripotent stem cells.

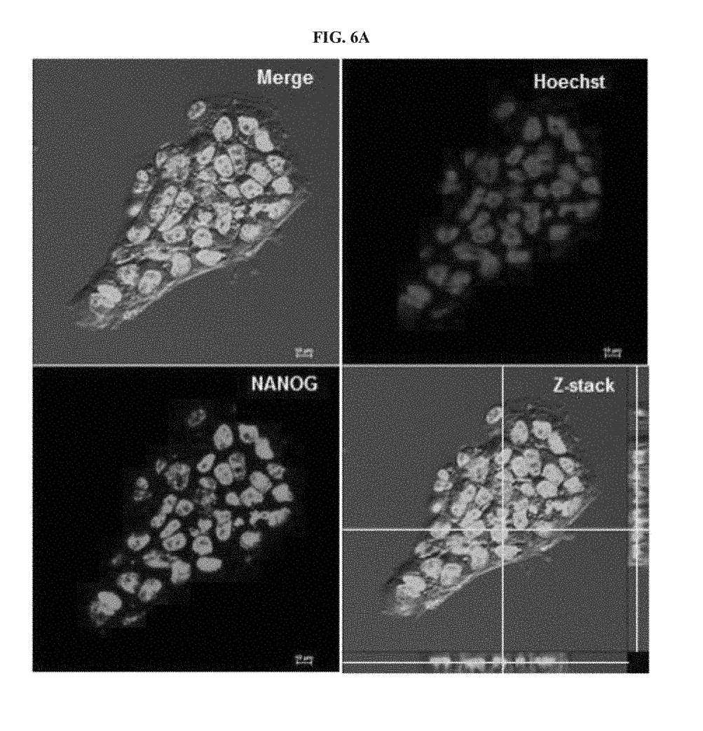

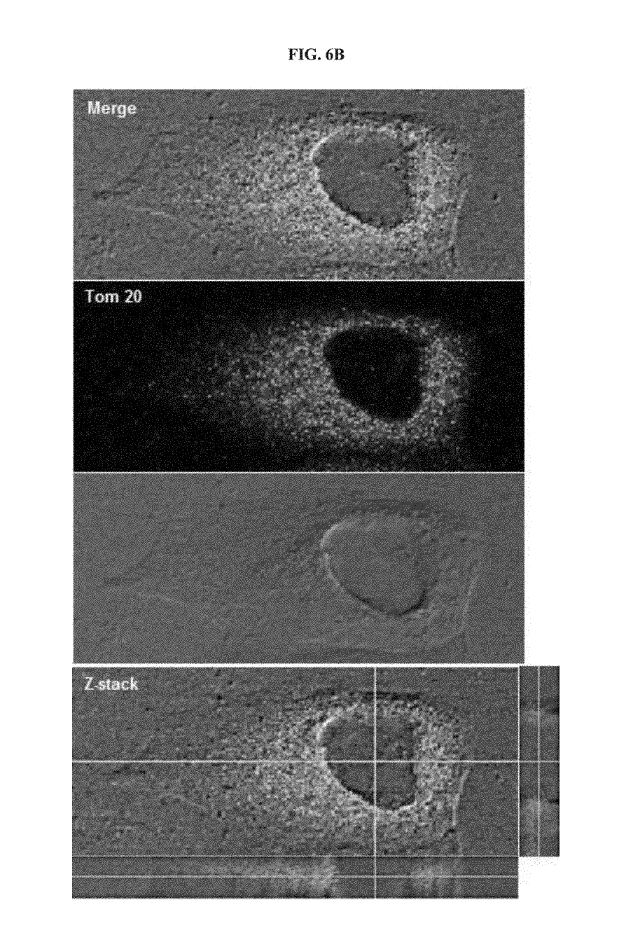

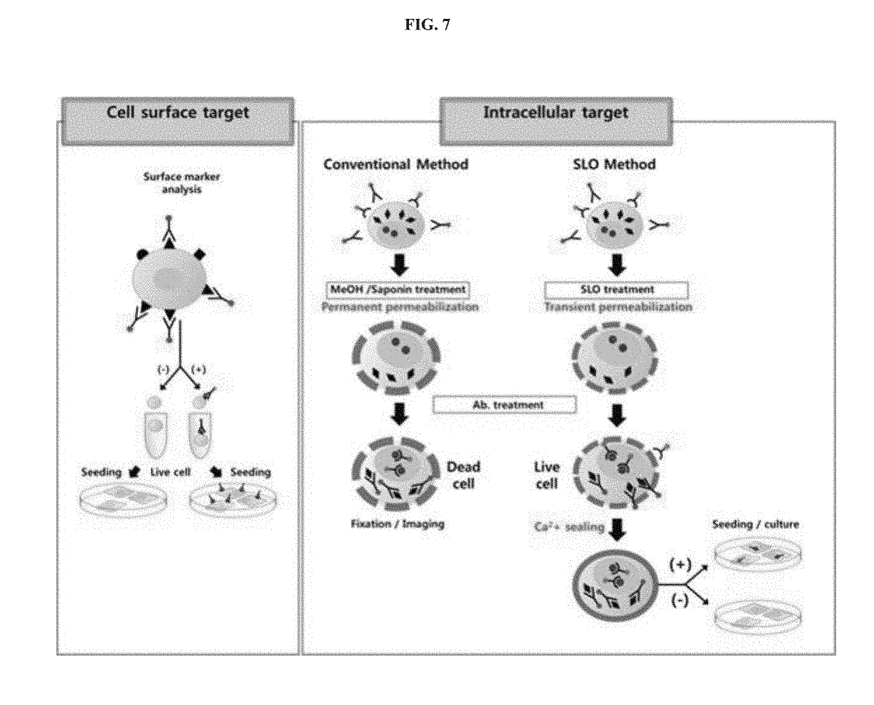

Afterward, the prepared human induced pluripotent stem cells were treated with 5 U SLO, thereby obtaining permeabilized cells. A 1:100 dilution of mouse-anti-NANOG (Cell Signaling) was incubated at 37□, a 1:100 dilution of secondary antibodies, i.e., anti-mouse-Alexa Fluor 488, was incubated at 37□. To regenerate the cell membrane of a cell, the stem cells which had been subjected to the antibody reaction were seeded in a Matrigel coated dish after 2 mM CaCl₂) was added to the same volume of hES media. As shown in FIG. 6A, confocal imaging was conducted for the cells in a living state, showing that the nucleus was stained by the transcription factor NANOG, and through Z-stack, it was demonstrated that staining is carried out using NANOG which has permeated a cell membrane.

### Example 6-2. Differentiation of Fibroblast-iPSC Into Cardiomyocytes and Confirmation of Staining with Tom 20

For differentiation into cardiomyocytes, an ES medium of an embryonic body of fibroblast-iPSC during culture was removed, 0.5 mg/mL of dispase was dissolved in a bFGF-free ES medium, the cells were treated with 1 mL of the resulting ES medium and then incubated at 37□ for 1 hour, such that fibroblast-iPSC colonies were separated from feeder cells. Suspended fibroblast-iPSC colonies were harvested and transferred to a 15-mL tube, and then the fibroblast-iPSCs were washed twice with a bFGF-free ES medium. On the following day, 3 μM of CHIR99021, 25 ng/mL of BMP4, 50 μg/mL of vitamin C, and 100 ng/mL of Activin A were added to an RPMI1640 medium containing B27-minus insulin (RPMI1640+B27 minus insulin), and the fibroblast-iPSC colonies were incubated for exactly 24 hours. Afterward, 10 ng/mL of BMP4 and 10 ng/mL of VEGF, which were diluted concentrations, were added to the RPMI1640+B27 minus insulin medium, followed by further incubation for 3 days, and then the cells were rinsed with the RPMI1640+B27 minus insulin, followed by further incubation for 1 day. On the following day, 5 μM IWP2, which is a low molecular substance for inhibiting a Wnt signaling pathway, was added to the cells, followed by culturing for 2 days and continuous culturing in a RPMI1640+B27 medium, thereby obtaining beating cardiomyocytes (CMCs). The obtained CMCs were treated with 5 U SLO, thereby obtaining permeabilized cells, and the permeabilized cardiomyocytes were treated with mouse-anti-Tom20 (Santa Cruz), which is a representative marker of mitochondria, in a 1:100 dilution at 37□ for 30 minutes, a secondary antibody, anti-mouse-Alexa Fluor 488, was incubated in a 1:100 dilution at 37□ for 30 minutes. To regenerate the cell membrane of the cardiomyocytes which had been subjected to the antibody reaction, 2 mM CaCl₂) was added to the same volume of the cell culture solution, followed by incubation at 37□. As shown in FIG. 6A, the cardiomyocytes were visualized by live imaging in a living state, confirming that mitochondria were stained with Tom20, and through Z-stack, it was demonstrated that the cells were stained with Tom20 which had permeated through the cell membrane. The results are illustrated in FIG. 6B.

According to the method of the present invention, although a target protein is present in the cell, it can be detected and analyzed by a simple method using an antibody. In addition, isolation and culturing can be performed easily using a flow cytometer, and further, specific cells which are isolated and cultured can be attached to enable subculture and cryopreservation. In addition, conventionally, cells were able to be isolated and cultured only by targeting an extracellular membrane protein, but according to the method of the present invention, target cells of interest can be obtained more rapidly and accurately by forming a pore in various cell membranes using a streptococcal hemolytic exotoxin only with a frequently used flow cytometer and antibodies without limiting a specific protein target. Moreover, while, in the case of primary culture, cells can be isolated and cultured by targeting only a specific extracellular membrane protein with several complicated steps for a long time, the most important thing is, when the method of the present invention is used, homogeneous cells of interest, as well as various types of cells, can be obtained with a higher purity and a higher efficiency than the conventional method by targeting both the extracellular membrane and a specific intracellular protein without using complicated steps and taking a long time in primary culture. In addition, the present invention is a technique of isolating, analyzing and culturing desired cells by permeating an intracellular protein into the living cells using a commercially available monoclonal antibody without the manufacture of a probe such as a molecular beacon. By using these advantages, cells having various intracellular proteins, which were limited in research since they could not be isolated and cultured from a patient's blood or various tissues in the past, can be easily detected, isolated and cultured, and the present invention is expected to highly contribute to research on the mechanisms of various diseases and treatment thereof, as well as various applications targeting a specific intracellular protein.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1. A method of isolating target cells in a living state, comprising:
(s1) treating a test material including target cells with a streptococcal hemolytic exotoxin, i.e., Streptolysin O (SLO);
(s2) treating the test material with an antibody against a target intracellular protein of the target cells; and
(s3) isolating the target cells in a living state, which have a positive response to the antibody.

2. The method of claim 1, wherein the target cells include peripheral blood mononuclear cells (PBMCs), skeletal myoblasts (SMBs), smooth muscle cells (SMCs) and human induced pluripotent stem cells.

3. The method of claim 1, wherein, in the step (s1), the streptococcal hemolytic exotoxin, i.e., SLO, is treated at a concentration of 5 U/ml or less.

4. The method of claim 1, wherein, in the step (s2), the target intracellular protein is a marker protein of the target cells.

5. The method of claim 4, wherein the marker protein of the target cells includes calponin, smooth muscle actin (SMA), nuclear factor of activated T-cells, cytoplasmic 1 (NFATc1) and dextran.

6. The method of claim 4, wherein the marker protein of the target cells is NANOG for human induced pluripotent stem cells or Tom20 of cardiomyocytes.

7. A method of isolating target cells in a living state, comprising:
(s1) treating a test material including target cells with a streptococcal hemolytic exotoxin, i.e., Streptolysin O (SLO);
(s2) treating the test material with an antibody against a target intracellular protein of the target cells; and
(s3) isolating the target cells in a living state, which have a positive response to the antibody through fluorescence-activated cell sorting (FACS) analysis.

8. The method according to claim 7, wherein, in the step (s2), a fluorescent dye-conjugated antibody is used.

9. The method according to claim 7, wherein, in the step (s2), an antibody against target intracellular protein of the target cells is used as a primary antibody, and a fluorescent dye-conjugated secondary antibody is used.

10. The method according to claim 9, wherein the fluorescent dye-conjugated secondary antibody has a size of 150 kDa or less.

11. The method according to claim 7, wherein the fluorescent dye is one or more selected from the group consisting of FITC, PE, Alexa Fluor 488, DyLight 488, PerCP, PerCP-Cy5.5, Alexa Fluor 555, Alexa Fluor 633, Alexa Fluor 700, Alexa Fluor 405, Cy3 and Cy5.

12. The method according to claim 1, further comprising suspending the target cells in a calcium chloride (CaCl2)-added cell culture to regenerate a cell membrane.

13. The method according to claim 7, further comprising suspending the target cells in a calcium chloride (CaCl2)-added cell culture to regenerate a cell.
